(19) United States
(12) Patent Application Publication
(10) Pub. No.: US 2009/0036853 A1
(43) Pub. Date: Feb. 5, 2009

(54) Gel-forming polymer in an absorbent article

(76) Inventor: George Graham Allan, Kenmore, WA (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/888,555

(22) Filed: Aug. 1, 2007

(51) Int. Cl. A61F 13/15; A01K 61/00

(52) U.S. Cl. 604/368; 119/215

(57) ABSTRACT

A method for acquiring a gel-forming polymer and an absorbent article containing the gel-forming polymer. In one embodiment, the method includes the steps of providing a Hagfish, stimulating the Hagfish to excrete a gel-forming polymer, and drying the gel-forming polymer. In another embodiment, the method for acquiring a gel-forming polymer includes the steps of providing a Hagfish, placing the Hagfish in a container of water, stimulating the Hagfish to excrete a gel-forming polymer into the water, and drying the gel-forming polymer.

### FIELD OF THE INVENTION

The present invention relates to gel-forming polymers.

### BACKGROUND OF THE INVENTION

Superabsorbent polymers are widely used in absorbent articles including diapers, adult incontinence products, and sanitary napkins. Superabsorbent polymers are also are used in other applications as a drying agent.

One superabsorbent polymer thought to be useful is comprised of cross-linked polyacrylic acid that is partly neutralized. By neutralizing the cross-linked polyacrylic acid, osmotic sites can be generated. The absorbency can depend upon the concentration of immobilized ionic sites within the three-dimensional polymer network and the osmotic pressure the immobilized ionic sites can create within the polymer network. When placed in aqueous fluid such as water, 1 g of superabsorbent polymers comprised of polyacrylic acid can swell and can immobilize as much as 400 g of water.

Absorbent articles such as diapers and sanitary napkins are designed to absorb body exudates including, but not limited to, urine, menses, vaginal discharge, bowel movement material, and sweat. Liquids such as urine have osmotic potential as a result of the salt content of the liquid. In a liquid such as urine, 1 g of superabsorbent polymer comprised of polyacrylic acid may be able to immobilize only 40 g of urine due to the adverse effects of osmotic potential of the urine on the absorbent capacity of the superabsorbent polymer.

Naturally occurring superabsorbent polymers can be found in nature. Hagfish, a species of the Myxinidae family found in the waters of the Pacific Northwest, excrete a gel-forming polymer when stimulated or agitated. The gel-forming polymer has the capacity to gel seawater, which has osmotic potential.

There is a continuing unaddressed need for a method for acquiring the gel-forming polymer from Hagfish and processing the gel-forming polymer into a form useful in commercial applications such as absorbent articles and drying agents. There is a further unaddressed need for absorbent articles that include gel-forming polymer from Hagfish.

### SUMMARY OF THE INVENTION

A method for acquiring a gel-forming polymer is disclosed. In one embodiment, the method comprises the steps of providing a Hagfish, stimulating the Hagfish to excrete a gel-forming polymer, and drying the gel-forming polymer.

In another embodiment, the method for acquiring a gel-forming polymer comprises the steps of providing a Hagfish, placing the Hagfish in a container of water, stimulating the Hagfish to excrete a gel-forming polymer into the water, and drying the gel-forming polymer.

In either of the above embodiments, the method can further comprise the step of water washing the gel-forming polymer. In either of the above embodiments, the gel-forming polymer can be dried under reduced pressure. In either of the above embodiments, the gel-forming polymer can be dried by the process of solvent exchange with ethanol. In either of the above embodiments, the gel-forming polymer can be air dried.

An absorbent article can comprise a gel-forming polymer, wherein the gel-forming polymer is an excretion from a Hagfish. The absorbent article can comprise a topsheet and a backsheet and the gel-forming polymer can be between the topsheet and the backsheet. The absorbent article can further comprise an absorbent core between the topsheet and the backsheet. The gel-forming polymer can be between the topsheet and the absorbent core. The gel-forming polymer can be between the backsheet and the absorbent core. The gel-forming polymer can be in the absorbent core. The gel-forming polymer can be in the absorbent article in a dry granular condition. The absorbent article can be of a type selected from the group consisting of a diaper, an adult incontinence product, a sanitary napkin, a panti-liner, a wipe, and a tissue towel.

### BRIEF DESCRIPTION OF THE DRAWINGS

FIG. 1 is a flow diagram of a method for acquiring a gel-forming polymer.

FIG. 2 is a flow diagram of another embodiment of a method for acquiring a gel-forming polymer.

FIG. 3 is a schematic of an absorbent article.

FIG. 4 is a schematic of a tissue towel.

### DETAILED DESCRIPTION OF THE INVENTION

Hagfish occur in the waters of the Pacific Northwest and Atlantic Northeast and can be captured alive. An enclosure having small perforations and dead fish contained therein as bait can be lowered to the sea floor. Hagfish can be found on the sea floor at depths from about 100 to about 200 ft. Depending on the environmental conditions, Hagfish can be found at other depths. Hagfish can enter the enclosure to feed on the bait. Once the Hagfish has entered the enclosure, the enclosure can be hoisted to the surface and the seawater therein can be drained away through the perforations in the enclosure, thereby leaving the Hagfish in the enclosure without seawater. Hagfish can also be raised in a cultured environment and captured using methods known in the art.

Captured Hagfish can be stimulated to excrete a gel-forming polymer. The gel-forming polymer can be collected and the Hagfish, which is unharmed by the process, can then be transferred back to seawater or to a cultured environment. The gel-forming polymer can be collected by hand, by hand with the assistance of tools and/or machines, and in an automated or semi-automated process.

One method for stimulating Hagfish to excrete the gel-forming polymer is to anesthetize the Hagfish, wipe the Hagfish dry, and apply a mild electrical impulse (about 24 V) to the slime glands of the Hagfish. The electrical impulse stimulates excretion of the gel-forming polymer. The gel-forming polymer can be collected with a spatula. Excretion can also be stimulated by physically handling, jarring, touching, or otherwise agitating the Hagfish.

The excretion by Hagfish is thought to be comprised of a mucus-forming polymer and nanofibers up to 60 cm long. The nanofibers can have a tapered and elliptical cross-section. The maximum dimension of the cross-section is about 1.5 μm. The nanofiber produced by Hagfish is proteinaceous with amino acids threonine and serine comprising a portion of the chemical makeup. The proteins carry pendant hydroxyl groups capable of hydrogen bonding to water. The nanofibers are hydrophilic and have a large surface area. Part of the protein structure comprises amide groups in the immediate vicinity of the hydroxyl groups derived from the serine and threonine backbone units such that a variety of cyclic structures capable of immobilizing water are possible.

The mucus component may have a proteinaceous structure (about 77% by weight) comprising the amino acids praline, threonine, valine, and other amino acids. The mucus component may comprise amino (about 8.7% by weight) and neutral (about 2% by weight) sugars. Sulphate groups (about 6.2% by weight) can also be present. The mucus component can immobilize water without the presence of the nanofibers. The nanofibers may act as mechanical reinforcement and hold the mucus together in seawater.

Without being bound by theory, the gel-forming polymer excreted by Hagfish, which can effectively immobilize seawater having osmotic potential, is thought to have efficacy with respect to immobilizing body exudates such as urine, blood, menses, sweat, and bowel movement material, which also can have osmotic potential. Without being bound by theory, it is thought that the gel-forming polymer excreted by Hagfish may be a more effective superabsorbent than superabsorbent polymers that are presently commercially available. Dried gel-forming polymer collected from Hagfish may be useful as a superabsorbent polymer or material in absorbent articles including, but not limited to, diapers, adult incontinence products, sanitary napkins, wound dressings, and similar absorbent articles. Dried gel-forming polymer collected from Hagfish may also be useful as a drying agent or desiccant. Furthermore, gel-forming polymer excreted by Hagfish may also be useful for removing inorganic and organic chemicals, from air, water, sediments, and soil. The gel-forming polymer excreted by Hagfish may also be useful for removing heavy metals, oil, and other hydrocarbons released into the air, water, sediments, and soil. The gel-forming polymer excreted by Hagfish may also be useful for cleaning up oil from oil slicks that are sometimes created in seawater and freshwater.

The gel-forming polymer excreted by Hagfish may have a reduced tendency to gel-block. Gel-blocking is thought to occur as a result of the swelling and sealing action of the first fluid to reach the surface of the particle, which reduces the penetration and absorbency of a fluid into a gel-forming particle.

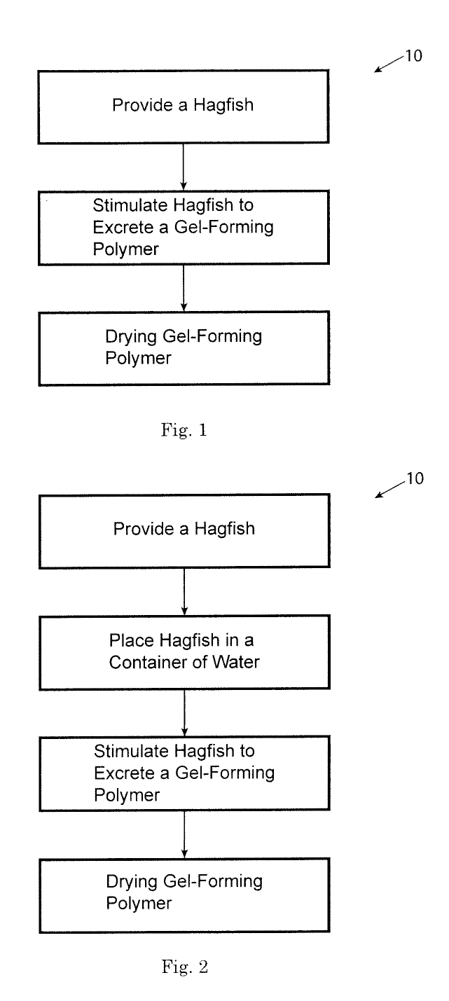

One embodiment of a method 10 for acquiring a gel-forming polymer is illustrated in FIG. 1. The method 10, shown in FIG. 1, comprises the steps of, providing a Hagfish, stimulating the Hagfish to excrete a gel-forming polymer, and drying the gel-forming polymer. The method 10 can further comprise the step of water washing the gel-forming polymer. The gel-forming polymer can be water washed until the gel-forming polymer is substantially free of salts. The step of drying the gel-forming polymer can be performed under reduced pressure. The step of drying the gel-forming polymer can be performed by solvent exchange. The step of drying the gel-forming polymer can be performed by solvent exchange with ethanol. The step of drying the gel-forming polymer can be performed by air drying.

Another embodiment of a method 10 for acquiring a gel-forming polymer is illustrated in FIG. 2. The method 10, shown in FIG. 2, comprises the steps of, providing a Hagfish, placing the Hagfish in a container of water, stimulating the Hagfish to excrete a gel-forming polymer, and drying the gel-forming polymer. The water in the container can be selected from the group consisting of seawater, freshwater, and mixtures thereof. The container can contain a liquid other than water in which the Hagfish can be stimulated to excrete the gel-forming polymer. When the Hagfish excretes gel-forming polymer into the container, a portion of the seawater, or other liquid if used, can become gelled. The gelled portion of the sea water can be separated from the un-gelled seawater using common methods of separation known in the art. One method for separating the gelled portion of the sea water is to pass the gelled portion and un-gelled portions of the seawater through a fine nylon mesh to remove and collect the gelled portion.

The method 10 illustrated in FIG. 2 can further comprise the step of water washing the gel-forming polymer. The gel-forming polymer can be water washed until the gel-forming polymer is substantially free of salts. Gel-forming polymer excreted in the container of water can be dried using the same approaches as disclosed above.

Marine chordates, other than Hagfish, are contemplated to be within the scope of the present disclosure.

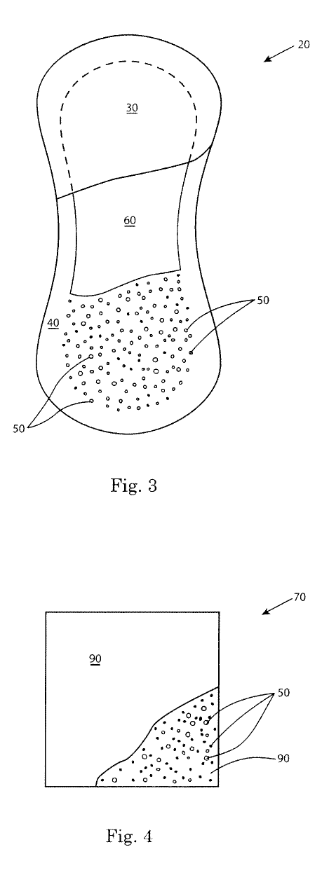

The gel-forming polymer 50 excreted by Hagfish is thought to be useful in an absorbent article 20, an example of which is shown in FIG. 3. Dry gel-forming polymer 50 can be formed into granules having a particle size less than about 1 mm. Dry gel-forming polymer 50 can be formed into granules having a particle size less than about 0.1 mm. The absorbent article 20 can comprise a topsheet 30 and a backsheet 40. An absorbent core 60 can be disposed between the topsheet 30 and backsheet 40. The gel-forming polymer 50 can be disposed between the topsheet 30 and backsheet 40. In one embodiment, the gel-forming polymer 50 can be between the topsheet 30 and absorbent core 60, as shown in FIG. 3. In another embodiment, the gel-forming polymer 50 can be disposed between the absorbent core 60 and the backsheet 40, as shown in FIG. 3. Gel-forming polymer 50 can also be disposed within the structure of the absorbent core 60. The absorbent article illustrated in FIG. 3 can be employed as a sanitary napkin, diaper, or adult incontinence product. An example of a diaper can be found in U.S. Pat. No. 5,968,025 issued to Roe et al. The topsheet 30 can be a hydroformed film, examples of which include that disclosed in U.S. Pat. No. 4,609,518 issued to Curro et al. on Sep. 2, 1986 and ALWAYS DRI-YWEAVE available from the Procter & Gamble Co., Cincinnati, Ohio.

A suitable absorbent core 60 can be comprised of multiple plies of creped cellulose wadding, fluffed cellulose fibers, wood pulp fibers also known as airfelt, textile fibers, a blend of fibers, a mass or batt of fibers, airlaid webs of fibers, a web of polymeric fibers, and a blend of polymeric fibers. The backsheet 40 can be comprised of polymer film, and film/non-woven laminates having a desirable vapor permeability. Other materials and components of absorbent articles 20 are disclosed in U.S. Pat. No. 4,950,264 issued to Osborn III Aug. 21, 1990 and U.S. Pat. No. 5,439,458 issued to Noel et al. Aug. 8, 1995.

Gel-forming polymer 50 is thought to be useful in absorbent articles selected from the group consisting of a diaper, an adult incontinence product, a sanitary napkin, a panti-liner, a wipe, and a tissue towel. Wipes can be form of cellulose-based materials or synthetic polymer materials wherein the fibers are arranged in either a woven or non-woven arrangement and can be either air-laid fibrous webs or wet-laid fibrous webs. Similarly, tissue towels can comprise one or more webs comprising cellulosic-fibers and/or synthetic fibers and the gel-forming polymer 50 can be positioned between two webs or within one or more of the fibrous webs. A tissue towel 70 comprising gel-forming polymer 50 between to cellulose-based webs 90 is illustrated in FIG. 4.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for acquiring a gel-forming polymer comprising the steps of:
providing a Hagfish;
stimulating said Hagfish to excrete a gel-forming polymer; and
drying said gel-forming polymer.

2. The method according to claim 1 further comprising the step of water washing said gel-forming polymer.

3. The method according to claim 1 wherein drying said gel-forming polymer is performed under reduced pressure.

4. The method according to claim 1 wherein drying said gel-forming polymer is performed by solvent exchange with ethanol.

5. The method according to claim 1 wherein drying said gel-forming polymer is performed by air drying.

6. A method for acquiring a gel-forming polymer comprising the steps of:
providing a Hagfish;
placing said Hagfish in a container of water;
stimulating said Hagfish to excrete a gel-forming polymer into said water; and
drying said gel-forming polymer.

7. The method according to claim 6 further comprising the step of water washing said gel-forming polymer.

8. The method according to claim 6 wherein drying said gel-forming polymer is performed under reduced pressure.

9. The method according to claim 6 wherein drying said gel-forming polymer is performed by solvent exchange with ethanol.

10. The method according to claim 6 wherein drying said gel-forming polymer is performed by air drying.

11. An absorbent article comprising a gel-forming polymer, wherein said gel-forming polymer is an excretion from a Hagfish.

12. The absorbent article of claim 11, wherein said absorbent article comprises a topsheet and a backsheet, wherein said gel-forming polymer is between said topsheet and said backsheet.

13. The absorbent article of claim 12 further comprising an absorbent core disposed between said topsheet and said backsheet.

14. The absorbent article of claim 13, wherein said gel-forming polymer is between said topsheet and said absorbent core.

15. The absorbent article of claim 13, wherein said gel-forming polymer is between said absorbent core and said backsheet.

16. The absorbent article of claim 13, wherein said gel-forming polymer is in said absorbent core.

17. The absorbent article of claim 11, wherein said gel-forming polymer is in a dry granular condition.

18. The absorbent article of claim 11, wherein said absorbent article is of a type selected from the group consisting of a diaper, an adult incontinence product, a sanitary napkin, a panti-liner, a wipe, and a tissue towel.
